(19) United States
(12) Patent Application Publication
(10) Pub. No.: US 2002/0058066 A1
(43) Pub. Date: May 16, 2002

(54) Cilostazol dry coated tablet

(75) Inventors: Yuso Tomohira, Tokushima-shi (JP); Daisuke Kuribayashi, Kakogawa-shi (JP); Junichi Jinno, Naruto-shi (JP); Yoshikazu Oka, Tokushima-shi (JP); Tadashi Mukai, Naruto-shi (JP)

Correspondence Address: Finnegan, Henderson, Farabow, Garrett & Dunner, L.L.P., 1300 I Street, N.W., Washington, DC, 20005-3315, US

(73) Assignee: Otsuka Pharmaceutical Co., Ltd.

(21) Appl. No.: 09/957,034

(22) Filed: Sep. 21, 2001

(30) Foreign Application Priority Data
Sep. 22, 2000 (JP) 2000-288023

(51) Int. Cl. A61K 9/36; A61K 31/47

(52) U.S. Cl. 424/480; 514/312

(57) ABSTRACT

Disclosed is a cilostazol dry coated tablet which comprises (A) a core portion containing cilostazol and (B) an outer layer portion containing cilostazol, a water-insoluble substance and a hydrophilic hydrogel forming substance. Said tablet is a new type of preparation capable of suppressing an undesirable rise of the maximum blood concentration of cilostazol while it maintains an adequate blood concentration of cilostazol by continuously releasing, for hours, only a required amount of cilostazol for obtaining a desired drug efficacy of cilostazol.

### BACKGROUND OF THE INVENTION

The present invention relates to a cilostazol dry coated tablet having excellent durability.

Cilostazol (general name of 6-[4-(1-cyclohexyl-1H-tetrazol-5-yl) butoxy]-3,4-dihydrocarbostyryl) shows not only a high platelet aggregation suppression action but also various kinds of medical actions such as a phosphodiesterase inhibition action, an anti-ulcer action, a hypotensive action and an anti-phlogistic action. Therefore, cilostazol has been widely used as thrombolytic drug, cerebral circulation improving drug, anti-phlogistic drug, anti-ulcer drug, hypotensive drug, drug for asthma, phosphodiesterase inhibitor and the like.

Cilostazol is usually used in the form of a tablet produced by adding excipients and other ingredients and compressing the mixture, and is orally administered. However, since the tablet is quickly disintegrated in the living body when orally administered, a large amount of cilostazol is released in the living body within a short time thereby to cause high blood concentration of cilostazol, resulting in side effects such as headache, dull headache, or pain.

### SUMMARY OF THE INVENTION

The present inventors have made various kinds of studies in order to solve the above-mentioned problems of the conventional cilostazol preparations and to develop a new type of a cilostazol preparation capable of suppressing an undesirable rise of the maximum blood concentration of cilostazol and maintaining an adequate blood concentration of cilostazol by continuously releasing, for hours, only a required amount of cilostazol for obtaining a desired drug efficacy of cilostazol. As a result, the inventors have found that making a cilostazol preparation into a specified dry coated tablet can solve the problems of the present invention. The present invention has been completed on the basis of this finding.

The present invention relates to a cilostazol dry coated tablet consisting of (A) a core portion containing cilostazol and (B) an outer layer portion coating the core portion and containing cilostazol, a water-insoluble substance and a hydrophilic hydrogel forming substance.

### BRIEF DESCRIPTION OF THE DRAWING

FIG. 1 is a graph showing relationship between the time and the dissolution rate of cilostazol evaluated by the dissolution tests according to sinker method and beads method.

### EMBODIMENTS OF THE INVENTION

Embodiments of a cilostazol dry coated tablet according to the present invention will be described in the following.

(1) A cilostazol dry coated tablet which comprises (A) a core portion containing cilostazol and (B) an outer layer portion containing cilostazol, a water-insoluble substance and a hydrophilic hydrogel forming substance.

(2) The cilostazol dry coated tablet according to the item (1), wherein the water-insoluble substance is ethylcellulose.

(3) The cilostazol dry coated tablet according to the item (1), wherein the hydrophilic hydrogel forming substance is hydroxypropylmethylcellulose.

(4) The cilostazol dry coated tablet according to the item (3), wherein the water-insoluble substance is ethylcellulose.

(5) The cilostazol dry coated tablet according to the items (1) to (4), wherein the outer layer portion further contains a water-very soluble substance.

(6) The cilostazol dry coated tablet according to the item (5), wherein the water-very soluble substance is D-mannitol.

(7) The cilostazol dry coated tablet according to the item (2), (4), (5) or (6), wherein the outer layer portion contains about 2 to 50% by weight of ethylcellulose.

(8) The cilostazol dry coated tablet according to the item (3), (4), (5) or (6), wherein the outer layer portion contains about 5 to 35% by weight of hydroxypropylmethylcellulose.

(9) The cilostazol dry coated tablet according to the item (4) or (5), wherein the outer layer portion contains about 2 to 50% by weight of ethylcellulose and about 5 to 35% by weight of hydroxypropylmethylcellulose.

(10) The cilostazol dry coated tablet according to the item (9), wherein the water-very soluble substance is D-mannitol and the outer layer portion contains about 3 to 20% by weight of D-mannitol.

(11) The cilostazol dry coated tablet according to the item (3), (4), (5), (7), (8), (9) or (10), wherein the viscosity of a 2% aqueous solution of hydroxypropylmethylcellulose at 20° C. is about 200 to 6000 cps.

(12) The cilostazol dry coated tablet according to the item (11), wherein the viscosity of a 2% aqueous solution of hydroxypropylmethylcellulose at 20° C. is about 200 to 1000 cps.

(13) The cilostazol dry coated tablet according to the item (12), wherein the outer layer portion contains about 5 to 50% by weight of ethylcellulose and about 5 to 35% by weight of hydroxypropylmethylcellulose.

(14) The cilostazol dry coated tablet according to the item (12), wherein the outer layer portion contains about 8 to 45% by weight of ethylcellulose, about 10 to 30% by weight of hydroxypropylmethylcellulose and about 3 to 20% by weight of D-mannitol.

(15) The cilostazol dry coated tablet according to the item (11), wherein the viscosity of a 2% aqueous solution of hydroxypropylmethylcellulose at 20° C. is about 3000 to 6000 cps.

(16) The cilostazol dry coated tablet according to the item (15), wherein the outer layer portion contains about 2 to 30% by weight of ethylcellulose and about 5 to 35% by weight of hydroxypropylmethylcellulose.

(17) The cilostazol dry coated tablet according to the item (15), wherein the outer layer portion contains about 5 to 25% by weight of ethylcellulose, about 10 to 30% by weight of hydroxypropylmethylcellulose and about 3 to 20% by weight of D-mannitol.

(18) The cilostazol dry coated tablet according to the item (2), (4), (6), (7), (9), (10), (13), (14), (16) or (17), wherein the proportion of the amount of ethylcellulose passing through an about 150 μm mesh sieve is less than about 70% by weight of the total amount of ethylcellulose.

(19) The cilostazol dry coated tablet according to the item (18), wherein the proportion of the amount of ethylcellulose passing through an about 75 μm mesh sieve is less than about 60% by weight of the total amount of ethylcellulose.

(20) The cilostazol dry coated tablet according to any of the items (1) to (19), wherein the core portion contains cilostazol, a hydrophilic hydrogel forming substance, a surfactant and a disintegrator.

(21) The cilostazol dry coated tablet according to any of the items (1) to (20), wherein the dissolution rates of cilostazol dissolved from the cilostazol dry coated tablet evaluated (a) in a dissolution test using a sinker according to the second method (paddle method) of the Japanese Pharmacopoeia dissolution test are about 5 to 25% after 2 hours, about 10 to 50% after 4 hours and more than about 40% after 10 hours, and (b) in a dissolution test using beads according to the second method (paddle method) of the Japanese Pharmacopoeia dissolution test are about 10 to 60% after 2 hours and more than about 25% after 4 hours.

(22) The cilostazol dry coated tablet according to any of the items (12) to (14), wherein the dissolution rates of cilostazol dissolved from the cilostazol dry coated tablet evaluated (a) in a dissolution test using a sinker according to the second method (paddle method) of the Japanese Pharmacopoeia dissolution test are about 5 to 15% after 2 hours, about 10 to 40% after 4 hours and more than about 40% after 10 hours, and (b) in a dissolution test using beads according to the second method (paddle method) of the Japanese Pharmacopoeia dissolution test are about 10 to 40% after 2 hours and more than about 25% after 4 hours.

(23) The cilostazol dry coated tablet according to any of the items (15) to (17), wherein the dissolution rates of cilostazol dissolved from the cilostazol dry coated tablet evaluated (a) in a dissolution test using a sinker according to the second method (paddle method) of the Japanese Pharmacopoeia dissolution test are about 5 to 25% after 2 hours, about 20 to 50% after 4 hours and more than about 50% after 10 hours, and (b) in a dissolution test using beads according to the second method (paddle method) of the Japanese Pharmacopoeia dissolution test are about 20 to 60% after 2 hours and more than about 60% after 4 hours.

The cilostazol dry coated tablet according to the present invention is a dry coated tablet preparation consisting of a core portion and an outer layer portion coating the core portion.

The cilostazol dry coated tablet according to the present invention has an important feature that the outer layer portion further contains a water-insoluble substance in addition to cilostazol and hydrophilic hydrogel forming substance.

The hydrophilic hydrogel forming substance is a high molecular substance, which swells in water to form gel. It includes, for example, a cellulose derivative such as methylcellulose, hydroxypropylcellulose (HPC), hydroxypropylmethylcellulose (HPMC), sodium carboxymethylcellulose, polyvinylalcohol or the like. Among these, HPMC is particularly preferred. They are used alone or in combination.

HPMC of different viscosities can be used. For example, a low viscosity HPMC, a 2% aqueous solution of which at 20° C. has a viscosity of about 200 to 1000 cps, a high viscosity HPMC, a 2% aqueous solution of which at 20° C. has a viscosity of about 3000 to 6000 cps, and the like can be used. Generally, when a high viscosity HPMC is used, the releasing rate of the active ingredient (cilostazol) from the preparation tends to be lowered, and on the contrary, when a low viscosity HPMC is used, the releasing rate of the same tends to be raised. Therefore, by suitably selecting HPMC having different viscosities and adequately combining them, the cilostazol releasing capability from the outer layer portion can be controlled.

The water-insoluble substance contained in the outer layer portion is a pharmaceutically acceptable and pharmacologically inert substance. The water-insoluble substance forms, together with the above-mentioned cilostazol and the hydrophilic hydrogel forming substance, a pH-independent matrix and gives strength to the outer layer portion, so that the outer layer portion can be gradually and stably disintegrated under the mechanically stimulating circumstances, for example, in a digestive tract.

The water-insoluble substance includes, for example, a specified pH-independent water-insoluble high molecular substances usually used as a base or a film of a sustained release preparation, and preferably, a water-insoluble methacrylate copolymer and a water-insoluble cellulose such as ethylcellulose or cellulose acetate can be used. Among these, ethylcellulose is particularly preferred. These are used alone or in combination.

Among various kinds of ethylcellulose, preferred is ethylcellulose in which the proportion of the amount of ethylcellulose passing through an about 150 μm mesh sieve is less than about 70% by weight of the total amount of the ethylcellulose. And particularly preferred is ethylcellulose in which the proportion of the amount of ethylcellulose passing through an about 75 μm mesh sieve is less than about 60% by weight of the total amount of the ethylcellulose. By using such ethylcellulose, the cilostazol dry coated tablet particularly excellent in durability can be obtained.

The cilostazol dry coated tablet according to the present invention hardly change its dissolution capability by a taker's eating and can continuously release cilostazol. The cilostazol dry coated tablet according to the present invention hardly receive mechanical stimulus naturally in a taker's vacant stomach, and even after eating, it hardly receive mechanical stimuli by eaten food, strong motility of the digestive tract and the like, so that cilostazol releasing can be largely controlled and rising of blood concentration of cilostazol can be suppressed.

According to the present invention, the outer layer potion can further contain a water-very soluble substance. Examples of the water-very soluble substance to be contained in the outer layer portion include succharides such as D-mannitol, D-sorbitol, D-xylitol, lactose, sucrose, glucose, anhydrous maltose, D-fructose, dextran; water-soluble high-molecular substances such as polyethyleneglycol and polyvinylpyrrolidone; surfactants such as polyoxyethylene polyoxypropylene glycol, polyoxyethylene sorbitan higher fatty acid ester; salts such as sodium chloride and magnesium chloride; organic acids such as tartaric acid and citric acid; amino acids such as glycine, β-alanine and lysine hydrochloride. They are used alone or in combination.

In the present invention, the outer layer portion can contain the hydrophilic hydrogel forming substance generally within the range of about 5 to 35% by weight, preferably about 7 to 33% by weight, more preferably about 10 to 30% by weight based on the total weight of the outer layer portion.

Further, in the present invention, the outer layer portion can contain the water-insoluble substance generally within the range of about 2 to 50% by weight, preferably about 5 to 45% by weight, more preferably about 5 to 40% by weight based on the total weight of the outer layer portion.

The optimum amount of the water-insoluble substance to be used sometimes changes in accordance with the kind and the like of the used hydrophilic hydrogel forming substance.

For example, when a low viscosity HPMC, a 2% aqueous solution of which at 20° C. has a viscosity of about 200 to 1000 cps, is used as the hydrophilic hydrogel forming substance, the water-insoluble substance (particularly ethylcellulose) is used within the range of generally about 5 to 50% by weight, preferably about 10 to 45% by weight, more preferably about 15 to 40% by weight based on the total weight of the outer layer portion. As the case may be, the water-insoluble substance (particularly ethylcellulose) is used within the range of generally about 5 to 50% by weight, preferably about 8 to 40% by weight, more preferably about 10 to 35% by weight based on the total weight of the outer layer portion.

Further as the case may be, the water-insoluble substance (particularly ethylcellulose) is used within the range of generally about 5 to 50% by weight, preferably about 8 to 45% by weight, more preferably about 10 to 40% by weight based on the total weight of the outer layer portion.

Further, when a high viscosity HPMC, a 2% aqueous solution of which at 20° C. has a viscosity of about 3000 to 6000 cps, is used as the hydrophilic hydrogel forming substance, the water-insoluble substance (particularly ethylcellulose) is used within the range of generally about 2 to 30% by weight, preferably about 5 to 25% by weight, more preferably about 5 to 20% by weight based on the total weight of the outer layer portion.

In this invention, the outer layer portion can contain the water-very soluble substance generally within the range of about 3 to 20% by weight, preferably about 5 to 18% by weight, more preferably about 5 to 15% by weight based on the total weight of the outer layer portion.

Further, the outer layer portion can contain at need, in addition to cilostazol, hydrophilic hydrogel forming substance, water-insoluble substance and water-very soluble substance, excipients such as starches including corn starch, potato starch, alpha starch, dextrin and carboxymethylstarch, inorganic salts including light anhydrous silicic acid, synthetic aluminum silicate, magnesium aluminometasilicate, calcium phosphate and calcium carbonate, oils and fats including paraffin, wax and higher fatty acid, and celluloses; disintegrators such as starches, croscarmellose sodium, sodium carboxymethyl starch, carboxymethylcellulose, calcium carboxymethylcellulose, low substituted hydroxypropylcellulose, crystalline cellulose and cross-linked polyvinylpyrrolidone; lubricants such as magnesium stearate, talc and synthetic aluminum silicate; solubilizing agents such as coloring agents including edible coloring matters; and the like.

The final composition of the outer layer portion according to the present invention is desirably adjusted so that the dissolution rates of cilostazol dissolved from a tablet having the same composition with the said outer layer portion evaluated (a) in a dissolution test using a sinker according to the second method (paddle method) of the Japanese Pharmacopoeia dissolution test (hereinafter referred to as sinker method) become about 5 to 25% after 2 hours, about 10 to 50% after 4 hours and more than about 40% after 10 hours, and (b) in a dissolution test using beads according to the second method (paddle method) of the Japanese Pharmacopoeia dissolution test (hereinafter referred to as beads method) are about 10 to 60% after 2 hours and more than about 25% after 4 hours.

As the case may be, the dissolution rates of cilostazol dissolved from a dry coated tablet according to the present invention evaluated (a) in a dissolution test using a sinker according to the second method (paddle method) of the Japanese Pharmacopoeia dissolution test (sinker method) are about 5 to 15% after 2 hours, about 10 to 30% after 4 hours and more than about 40% after 10 hours, and (b) in a dissolution test using beads according to the second method (paddle method) of the Japanese Pharmacopoeia dissolution test (beads method) are about 10 to 25% after 2 hours, about 25 to 45% after 4 hours and more than about 50% after 10 hours.

As the case may be, the dissolution rates of cilostazol dissolved from a dry coated tablet according to the present invention evaluated (a) in a dissolution test using a sinker according to the second method (paddle method) of the Japanese Pharmacopoeia dissolution test (sinker method) are about 5 to 15% after 2 hours, about 10 to 40% after 4 hours and more than about 50% after 10 hours, and (b) in a dissolution test using beads according to the second method (paddle method) of the Japanese Pharmacopoeia dissolution test (beads method) are about 10 to 40% after 2 hours and more than about 30% after 4 hours.

As the case may be, the dissolution rates of cilostazol dissolved from a dry coated tablet according to the present invention evaluated (a) in a dissolution test using a sinker according to the second method (paddle method) of the Japanese Pharmacopoeia dissolution test (sinker method) are about 5 to 15% after 2 hours, about 10 to 40% after 4 hours and more than about 50% after 10 hours, and (b) in a dissolution test using beads according to the second method (paddle method) of the Japanese Pharmacopoeia dissolution test (beads method) are about 10 to 40% after 2 hours and more than about 30% after 4 hours.

As the case may be, the dissolution rates of cilostazol dissolved from a dry coated tablet according to the present invention evaluated (a) in a dissolution test using a sinker according to the second method (paddle method) of the Japanese Pharmacopoeia dissolution test (sinker method) are about 5 to 25% after 2 hours, about 20 to 50% after 4 hours and more than about 50% after 10 hours, and (b) in a dissolution test using beads according to the second method (paddle method) of the Japanese Pharmacopoeia dissolution test (beads method) are about 20 to 60% after 2 hours and more than about 60% after 4 hours.

As the case may be, the dissolution rates of cilostazol dissolved from a dry coated tablet according to the present invention evaluated (a) in a dissolution test using a sinker according to the second method (paddle method) of the Japanese Pharmacopoeia dissolution test (sinker method) are about 5 to 15% after 2 hours, about 10 to 40% after 4 hours and about 40 to 95% after 10 hours, and (b) in a dissolution test using beads according to the second method (paddle method) of the Japanese Pharmacopoeia dissolution test (beads method) are about 10 to 40% after 2 hours and more than about 25% after 4 hours.

In this specification, sinker method and beads method are carried out under the following conditions respectively.

Sinker method: 700 ml of aqueous 0.45% sodium lauryl sulfate solution containing 5 g of sodium chloride was used as the dissolution test solution. One tablet was put into a sinker, and the dissolution test was carried out according to the second method (paddle method) of the Japanese Pharmacopoeia 13 dissolution test method at the paddle rotation speed of 50 rpm. Filtrated sample was assayed spectrophotometrically.

Beads method: 700 ml of aqueous 0.45% sodium lauryl sulfate solution containing 5 g of sodium chloride was used as the dissolution test solution and about 1500 polystyrene beads that has 6 mm in diameter were put into the same. One tablet was fixed on the front surface of the paddle in the rotational direction with a two side tape, and the dissolution test was carried out according to the second method (paddle method) of the Japanese Pharmacopoeia 13 dissolution test method at the paddle rotation speed of 50 rpm. Filtrated sample was assayed spectrophotometrically.

The core portion constituting a dry coated tablet according to the present invention contains cilostazol and a hydrophilic hydrogel forming substance.

The hydrophilic hydrogel forming substance includes, for example, hydrophilic polymers such as hydroxypropylcellulose, hydroxypropylmethylcellulose, polyvinylpyrrolidone, methylcellulose, and poly(ethylene oxide); and polymer saccharides such as carageenan, guar gum and gum arabic; and these hydrophilic hydrogel forming substances can be used alone or in combination. Among these hydrophilic hydrogel forming substances, hydroxypropylcellulose, hydroxypropylmethylcellulose, methylcellulose, and poly(ethylene oxide) are preferred, and hydroxypropylmethylcellulose is particularly preferred.

The core portion can contain the above-mentioned hydrophilic hydrogel forming substance generally within the range of about 5 to 90% by weight, preferably about 10 to 80% by weight, more preferably about 10 to 70% by weight based on the total weight of the core portion.

The core portion can contain a surfactant and a disintegrator in addition to the hydrophilic hydrogel forming substance.

As the surfactant, those can be widely used which have been conventionally known in this field. For example, they include alkyl sulfate salt such as sodium lauryl sulfate and magnesium lauryl sulfate; polyglyceryl fatty acid ester such as decaglyceryl monolaurate and decaglyceryl monomyristate; polyoxyethylene sorbitan fatty acid ester such as polyoxyethylene sorbitan monooleate; polyethylene glycol fatty acid ester such as polyoxyethylene monostearate; polyoxyethylene alkyl ether such as polyoxyethylene lauryl ether; polyoxyethylene castor oil and hydrogenated castor oil, such as polyoxyethylene hydrogenated castor oil; sucrose ester of fatty acid such as sucrose stearate ester and sucrose palmitate ester; and alkyl sulfate such as sodium lauryl sulfate and magnesium lauryl sulfate. These surfactants can be used alone or in combination.

As the disintegrator, those can be widely used which have been conventionally known in this field. They include, for example, law substituted hydroxypropylcellulose, croscarmellose sodium, crospovidone, and carboxymethyl starch sodium. Among these disintegrators, law substituted hydroxypropylcellulose and croscarmellose sodium are preferred, and law substituted hydroxypropylcellulose is particularly preferred.

Further, the core portion can contain at need, for example, excipients such as starches including corn starch, potato starch, alpha starch, dextrin, carboxymethylstarch, saccharides such as lactose, sucrose, glucose, mannitol, sorbitol, inorganic salts such as light anhydrous silicic acid, synthetic aluminum silicate, magnesium aluminometasilicate, calcium phosphate and calcium carbonate, oils and fats such as paraffin, wax and higher fatty acid, and celluloses; lubricants such as magnesium stearate, talc and synthetic aluminum silicate; and coloring agents such as edible coloring matters and the like.

The dry coated tablet according to the present invention can be produced, for example, by forming a core tablet constituting the core portion by a conventional method, and then coating the core tablet with the outer layer portion having the abovementioned composition using a press coater.

Further, the abovementioned core tablet may be coated with a thin film coating prior to coating with the outer layer portion. The film coating material may includes, for example, water-soluble coating materials of celluloses such as HPC, HPMC, hydroxyethylcellulose and methylhydroxyethylcellulose; enteric coating material of celluloses such as hydroxypropylmethylcellulosephthalate, hydroxypropylmethylcellulose acetate succinate, carboxymethylethylcellulose and cellulose acetate phthalate; and other enteric film coating materials such as methacrylic acid copolymer, shellac and the like.

Further, the dry coated tablet according to the present invention may be provided with at least one layer of film coating. Such a film coating may be, for example, water-soluble coating of celluloses. Among the water-soluble coating, HPMC is preferred from the point of film forming property. HPMC, a 2% aqueous solution of which at 20° C. has a viscosity less than about 100 cps, especially about 15 cps, is particularly preferred. It is possible to add, at need, a plasticizer such as polyethylene glycol to the film coating material.

The dry coated tablet according to the present invention can contain generally about 50 to 300 mg, preferably about 70 to 250 mg of cilostazol per tablet. The relative ratio between cilostazol amounts contained in the core portion and the outer layer portion is not limited but the weight ratio of cilostazol amount in the core portion/cilostazol amount in the outer layer portion can be within the range of generally 10/90 to 95/5, preferably 20/80 to 90/10, more preferably 30/70 to 80/20.

The outer layer requires enough thickness to avoid erosion of the core tablet, and the thickness is not less than 1 mm, preferably not less than 1.5 mm, and more preferably about 1.5 to 3 mm, at one side.

After the dry coated tablet according to the present invention is orally administered, the outer layer portion absorbs digestive fluid and water at the upper portion of the digestive tract and forms an erodible matrix, but prevents rapid erosion even by motility of the digestive tract, because the dry coated tablet has a mechanical strength owing to the abovementioned structure. The outer layer gradually forms gel surely from outside, and stably releases cilostazol from the surface with time. Then, when the releasing of the drug from the matrix outer layer is substantially completed, the preparation reaches the lower portion of the digestive tract where there is small amount of water, and the core portion begins to release cilostazol.

Therefore, the dry coated tablet of the present invention is provided with an improved stability against the strong vermiculation and mechanical motility of the digestive tract, and has an advantage of maintaining the effective blood concentration of cilostazol by administering only one time per day.

The cilostazol dry coated tablet of the present invention is such a new type of preparation capable of suppressing an undesirable rise of the maximum blood concentration of cilostazol while it maintains the adequate blood concentration of cilostazol by continuously releasing, for hours, only required amount of cilostazol to obtain a desired drug efficacy of cilostazol.

Since the cilostazol dry coated tablet according to the present invention is not quickly disintegrated in the living body, a large amount of cilostazol is not released in the living body within a short time. Therefore, high blood concentration of cilostazol is not caused and side effects such as headache, dull headache, or pain can be substantially prevented.

The present invention will become more apparent from the following examples.

### EXAMPLE 1

1600 g of cilostazol, 300 g of sodium lauryl sulfate (surfactant, manufactured by NIKKO Chemicals Co.), 400 g of low-substituted hydroxypropylcellulose (LH-31, manufactured by SHINETSU Chemical Industries, Co., Ltd.), 120 g of hydroxypropylmethylcellulose (METOLOSE90SH400, manufactured by SHINETSU Chemical Industries, Co., Ltd.), 80 g of crystalline cellulose (Avicel PH301, manufactured by Asahi Kasei CO.), 78 g of hydroxypropylcellulose (HPC-L, manufactured by Nippon soda Co., Ltd.), and 10 g of magnesium oxide (manufactured by KYOWA Chemical Industry Co., Ltd.) were mixed and granulated with adding purified water to obtain granules for compression. Then, Mg stearate (manufactured by TAIHEI Chemical Industry Co. Ltd.) was added and the granules were compressed so that one core tablet had a weight of 130 mg using a punch having a diameter of 7 mm.

On the other hand, 960 g of cilostazol, 800 g of hydroxypropylmethylcellulose (METOLOSE90SH400, manufactured by SHINETSU Chemical Industries, Co., Ltd.), 400 g of D-mannitol (manufactured by KYOWA HAKKO KOGYO Co., Ltd.), 1200 g of ethylcellulose (ETHOCEL 7cps STANDARD PREMIUM, manufactured by DOW Chemical Co., Ltd.) and 616 g of lactose were mixed and granulated with adding purified water to obtain granules for compression. Then, Mg stearate was added and the granules were compressed using a punch to obtain dry coated tablets having a diameter of 11 mm and a weight of 630 mg containing the abovementioned core tablet.

### EXAMPLE 2

In the same manner as in Example 1, except that 800 g of ethylcellulose (ETHOCEL 7CPS STANDARD PREMIUM, manufactured by DOW Chemical Co., ltd.) and 616 g of lactose were used, granules for compression were obtained. Further, in the same manner as in Example 1, dry coated tablets having a diameter of 11 mm and a weight of 630 mg were obtained.

### EXAMPLE 3

In the same manner as in Example 1, except that 400 g of ethylcellulose (ETHOCEL 7CPS STANDARD PREMIUM, manufactured by DOW Chemical Co., Ltd.) and 1416 g of lactose were used, granules for compression were obtained. Further, in the same manner as in Example 1, dry coated tablets having a diameter of 11 mm and a weight of 630 mg were obtained.

Formulas in Examples 1 to 3 are shown in Table 1.

TABLE 1

| | Example 1 | Example 2 | Example 3 |
|---|---|---|---|
| Cilostazol | 80 | 80 | 80 |
| Sodium lauryl sulfate | 15 | 15 | 15 |
| HPC (LH-31) | 20 | 20 | 20 |
| HPMC(METOLOSE 90SH400) | 6 | 6 | 6 |
| Crystalline cellulose | 4 | 4 | 4 |
| HPC(HPC-L) | 3.9 | 3.9 | 3.9 |
| Mg oxide | 0.5 | 0.5 | 0.5 |
| Mg stearate | 0.6 | 0.6 | 0.6 |
| Core tablet(mg) | 130.0 | 130.0 | 130.0 |
| Cilostazol | 120 | 120 | 120 |
| HPMC(METOLOSE90SH400) | 100 | 100 | 100 |
| D-mannitol | 50 | 50 | 50 |
| Ethylcellulose | 150 | 100 | 50 |
| Lactose | 77 | 127 | 177 |
| Mg stearate | 3 | 3 | 3 |
| Total outer layer(mg) | 500 | 500 | 500 |
| Dry coated tablet(mg) | 630.0 | 630.0 | 630.0 |

### EXAMPLES 4 to 6

In the same manner as in Example 1, except that hydroxypropylmethylcellulose (METOLOSE90SH4000 manufactured by SHINETSU Chemical Industries, Co., Ltd.) was used instead of hydroxypropylmethylcellulose (METOLOSE90SH400 manufactured by SHINETSU Chemical Industries, Co., Ltd.), granules for compression were obtained. Further, in the same manner as in Example 1, dry coated tablets having a diameter of 11 mm and a weight of 630 mg were obtained.

Formulas in Examples 4 to 6 are shown in Table 2.

TABLE 2

| | Example 1 | Example 2 | Example 3 |
|---|---|---|---|
| Cilostazol | 80 | 80 | 80 |
| Sodium lauryl sulfate | 15 | 15 | 15 |
| HPC (LH-31) | 20 | 20 | 20 |
| HPMC(METOLOSE90SH400) | 6 | 6 | 6 |
| Crystalline cellulose | 4 | 4 | 4 |
| HPC(HPC-L) | 3.9 | 3.9 | 3.9 |
| Mg oxide | 0.5 | 0.5 | 0.5 |
| Mg stearate | 0.6 | 0.6 | 0.6 |
| Core tablet(mg) | 130.0 | 130.0 | 130.0 |
| Cilostazol | 120 | 120 | 120 |
| HPMC(METOLOSE90SH4000) | 75 | 75 | 75 |
| D-mannitol | 50 | 50 | 50 |
| Ethylcellulose | 150 | 100 | 50 |
| Lactose | 102 | 152 | 202 |
| Mg stearate | 3 | 3 | 3 |
| Total outer layer(mg) | 500 | 500 | 500 |
| Dry coated tablet(mg) | 630.0 | 630.0 | 630.0 |

### TEST EXAMPLE 1

Predetermined amounts of cilostazol, hydroxypropylmethylcellulose (METOLOSE90SH400, manufactured by SHINETSU Chemical Industries, Co., Ltd.) and ethylcellulose (sieved through a 500 μm mesh screen. ETCEL 7cps STANDARD PREMIUM, manufactured by DOW Chemical Co., Ltd.) were mixed and granulated with adding purified Water to obtain granules. The granules were sieved through a 850 μm mesh screen. Then, Mg stearate was added and the granules were compressed using a punch having a diameter of 11 mm to obtain a tablet having and a weight of 500 mg. 120 mg of cilostazol, 100 mg of hydroxypropylmethylcellulose and 277 g of ethylcellulose and 3 mg of Mg stearate were contained in one tablet.

Dissolution test of this tablet was carried out.

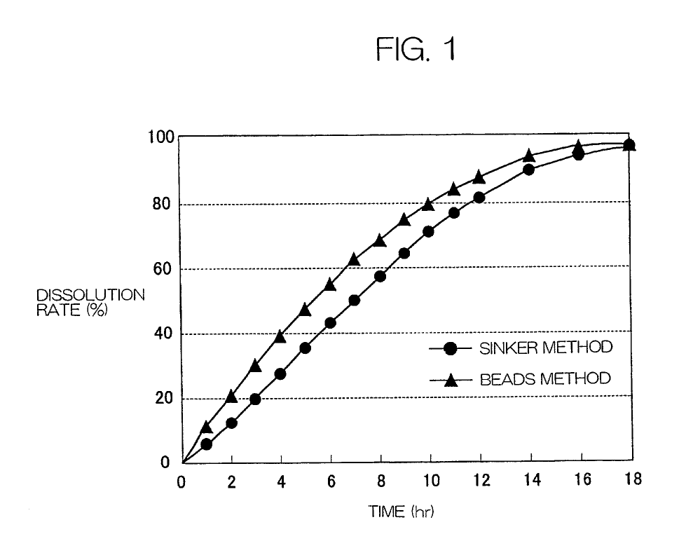

In the dissolution test, sinker method and beads method were applied. In both the tests of sinker method and beads method, the dissolution rates of cilostazol were evaluated at intervals of an hour from the beginning of the test to 18 hours after. The results are shown in FIG. 1.

What is claimed is:

1. A cilostazol dry coated tablet which comprises (A) a core portion containing cilostazol and (B) an outer layer portion containing cilostazol, a water-insoluble substance and a hydrophilic hydrogel forming substance.

2. The cilostazol dry coated tablet according to claim 1, wherein the water-insoluble substance is ethylcellulose.

3. The cilostazol dry coated tablet according to claim 1, wherein the hydrophilic hydrogel forming substance is hydroxypropylmethylcellulose.

4. The cilostazol dry coated tablet according to claim 3, wherein the water-insoluble substance is ethylcellulose.

5. The cilostazol dry coated tablet according to claim 4, wherein the outer layer portion further contains water-very soluble substance.

6. The cilostazol dry coated tablet according to claim 5, wherein the water-very soluble substance is D-mannitol.

7. The cilostazol dry coated tablet according to claim 2, wherein the outer layer portion contains about 2 to 50% by weight of ethylcellulose.

8. The cilostazol dry coated tablet according to claim 3, wherein the outer layer portion contains about 5 to 35% by weight of hydroxypropylmethylcellulose.

9. The cilostazol dry coated tablet according to claim 4, wherein the outer layer portion contains about 2 to 50% by weight of ethylcellulose and about 5 to 35% by weight of hydroxypropylmethylcellulose.

10. The cilostazol dry coated tablet according to claim 9, wherein the outer layer portion contains about 3 to 20% by weight of D-mannitol.

11. The cilostazol dry coated tablet according to claim 3, wherein the viscosity of a 2% aqueous solution of hydroxypropylmethylcellulose at 20° C. is about 200 to 6000 cps.

12. The cilostazol dry coated tablet according to claim 11, wherein the viscosity of a 2% aqueous solution of hydroxypropylmethylcellulose at 20° C. is about 200 to 1000 cps.

13. The cilostazol dry coated tablet according to claim 12, wherein the outer layer portion contains about 5 to 50% by weight of ethylcellulose and about 5 to 35% by weight of hydroxypropylmethylcellulose.

14. The cilostazol dry coated tablet according to claim 12, wherein the outer layer portion contains about 8 to 45% by weight of ethylcellulose, about 10 to 30% by weight of hydroxypropylmethylcellulose and about 3 to 20% by weight of D-mannitol.

15. The cilostazol dry coated tablet according to claim 11, wherein the viscosity of a 2% aqueous solution of hydroxypropylmethylcellulose at 20° C. is about 3000 to 6000 cps.

16. The cilostazol dry coated tablet according to claim 15, wherein the outer layer portion contains about 2 to 30% by weight of ethylcellulose and about 5 to 35% by weight of hydroxypropylmethylcellulose.

17. The cilostazol dry coated tablet according to claim 15, wherein the outer layer portion contains about 5 to 25% by weight of ethylcellulose, about 10 to 30% by weight of hydroxypropylmethylcellulose and about 3 to 20% by weight of D-mannitol.

18. The cilostazol dry coated tablet according to claim 2, wherein the proportion of the amount of ethylcellulose passing through an about 150 μm mesh sieve is less than about 70% by weight of the total amount of ethylcellulose.

19. The cilostazol dry coated tablet according to claim 18, wherein the proportion of the amount of ethylcellulose passing through an about 75 μm mesh sieve is less than about 60% by weight of the total amount of ethylcellulose.

20. The cilostazol dry coated tablet according to claim 1, wherein the core portion contains cilostazol, a hydrophilic hydrogel forming substance, a surfactant and a disintegrator.

21. The cilostazol dry coated tablet according to claim 1, wherein the dissolution rates of cilostazol dissolved from the cilostazol dry coated tablet evaluated (a) in a dissolution test using a sinker according to the second method (paddle method) of the Japanese Pharmacopoeia dissolution test are about 5 to 25% after 2 hours, about 10 to 50% after 4 hours and more than about 40% after 10 hours, and (b) in a dissolution test using beads according to the second method (paddle method) of the Japanese Pharmacopoeia dissolution test are about 10 to 60% after 2 hours and more than about 25% after 4 hours.

22. The cilostazol dry coated tablet according to claim 12, wherein the dissolution rates of cilostazol dissolved from the cilostazol dry coated tablet evaluated (a) in a dissolution test using a sinker according to the second method (paddle method) of the Japanese Pharmacopoeia dissolution test are about 5 to 15% after 2 hours, about 10 to 40% after 4 hours and more than about 40% after 10 hours, and (b) in a dissolution test using beads according to the second method (paddle method) of the Japanese Pharmacopoeia dissolution test are about 10 to 40% after 2 hours and more than about 25% after 4 hours.

23. The cilostazol dry coated tablet according to claim 15, wherein the dissolution rates of cilostazol dissolved from the cilostazol dry coated tablet evaluated (a) in a dissolution test using a sinker according to the second method (paddle method) of the Japanese Pharmacopoeia dissolution test are about 5 to 25% after 2 hours, about 20 to 50% after 4 hours and more than about 50% after 10 hours, and (b) in a dissolution test using beads according to the second method (paddle method) of the Japanese Pharmacopoeia dissolution test are about 20 to 60% after 2 hours and more than about 60% after 4 hours.
